(19) United States
(12) Patent Application Publication
(10) Pub. No.: US 2015/0018723 A1
(43) Pub. Date: Jan. 15, 2015

(54) APPARATUS FOR EARLY DETECTION OF PARALYSIS BASED ON MOTION SENSING

(71) Applicant: Industry-Academic Cooperation Foundation, Kyungpook National University, Daegu (KR)

(72) Inventors: Yang Soo Lee, Daegu (KR); Yang Ha Hwang, Daegu (KR)

(21) Appl. No.: 13/938,202

(22) Filed: Jul. 9, 2013

(51) Int. Cl. A61B 5/11

(52) U.S. Cl. CPC A61B 5/1118
USPC 600/595

(57) ABSTRACT

Disclosed herein is an apparatus for early detection of paralysis based on motion sensing. The apparatus includes: a sensing part which detects motion characteristic values of at least one left distal end and at least one right distal end of upper or lower limbs of a patient; a motion disorder determination unit which determines whether a difference between a sum of the motion characteristic values of the at least one left distal end and a sum of the motion characteristic values of the at least one right distal end, detected by the sensing part for a certain period of time, exceeds a reference value. The motion disorder determination unit determines whether a patient's motion is abnormal based on a difference in motion characteristics of distal ends, such as left and right wrists and ankles, and the like, for a certain period of time, thereby enabling detection of a stroke at an early stage. In addition, sensors of the sensing part may be attached to the hands and feet, and worn on the wrists and ankles like watches or anklets, thereby improving convenience.

### BACKGROUND

The present invention relates to an apparatus for early detection of paralysis, and more particularly, to an apparatus for early detection of paralysis based on motion sensing, which can detect a patient suffering paralysis, such as a stroke, at an early stage.

A stroke is caused by rupture or blockage of a blood vessel in the brain. It is already well known that treatment at an early stage is required to provide better treatment effects upon the occurrence of a stroke. Therefore, in order to rapidly determine whether a person suffers a stroke, rapid diagnosis of a patient suspected of having suffered a stroke is of utmost importance.

Particularly, patients recovered from a mild stroke have a high possibility of suffering from a recurrence of a severe stroke. If a recurring stroke of these patients can be detected and treated at an early stage, however, the treated patients would be highly likely to show a favorable prognosis. Generally, after the occurrence of a stroke, a patient shows a hemi-paralysis symptom that one-side limbs of the patient are paralyzed at an early stage. Occasionally, the patient shows a monoparesis symptom that only one arm or leg of the patient is paralyzed, or only both legs of the patient are paralyzed. However, there is no apparatus which can determine the occurrence of a stroke by detecting characteristics of a patient showing early-stage symptoms of stroke.

Therefore, there is an urgent need for an apparatus for early detection of disease, such as a stroke, which accompanies a severe and long-term disability and may lead to death, due to late treatment. Specific advantages and features of the invention will be apparent from the accompanying drawings and description of an illustrative embodiment of the present invention.

### BRIEF SUMMARY

The present invention has been conceived to solve the above problems, and it is an aspect of the present invention to provide an apparatus for early detection of paralysis based on motion sensing, which can detect the occurrence of a stroke at an early stage.

In accordance with one aspect of the present invention, an apparatus for early detection of paralysis based on motion sensing includes: a sensing unit which detects motion characteristic values of at least one left distal end and at least one right distal end of upper or lower limbs of a patient; a motion disorder determination unit which determines whether a difference between the characteristic values of the motion of the at least one left distal end and the at least one right distal end detected by the sensing unit for a certain period of time exceeds a reference value. At this time, the sensing unit may be placed at a distal portion of a patient body, such as wrists, and ankles.

The sensing unit may include a first communication unit transmitting the motion characteristic value.

The apparatus may further include an alarm unit which warns of a motion disorder when the motion disorder determination unit determines that the difference between the characteristic values of the motion of the at least one left distal end and the at least one right distal end exceeds the reference value.

The alarm unit may include a second communication unit transmitting a signal containing information regarding the motion disorder.

The apparatus may further include a display unit displaying the information of the motion disorder received from the second communication unit.

The motion characteristic value of each of the distal ends may include velocity, acceleration, or distance moved.

The at least one left distal end may correspond to the at least one right distal end.

In accordance with another aspect of the present invention, an apparatus for early detection of paralysis based on motion sensing includes: a sensing unit detecting a motion characteristic value of at least one distal end of upper or lower limbs; a motion disorder determination unit determining whether a difference between a sum of motion characteristic values of the at least one distal end detected by the sensing unit for a unit time and a sum of motion characteristic values of the at least one distal end detected by the sensing unit for a subsequent unit time exceeds a reference value.

The sensing unit may include a first communication unit transmitting the motion characteristic value.

The apparatus may further include an alarm unit which warns of a motion disorder when the motion disorder determination unit determines that the difference between the sum of the characteristic values of the motion of the at least one distal end detected for a unit time and the sum of characteristic values of the motion of the at least one distal end detected by the sensing unit for a subsequent unit time exceeds the reference value.

The alarm unit may include a second communication unit transmitting a signal containing information of the motion disorder.

The apparatus may further include a display unit displaying the information of the motion disorder received from the second communication unit.

The characteristic values of the motion of the distal end may include velocity, acceleration, or distance moved.

In accordance with a further aspect of the present invention, an apparatus for early detection of paralysis based on motion sensing includes: a sensing unit detecting a motion characteristic value of at least one distal end of upper or lower limbs; and a motion disorder determination unit determining whether a sum of motion characteristic values of the at least one distal end detected by the sensing unit for a unit time is less than a reference value.

The apparatus may further include an alarm unit warning about a motion disorder when the motion disorder determination unit determines that the sum of the motion characteristic values of the at least one distal end detected by the sensing unit for a unit time is less than the reference value.

The alarm unit may include a second communication unit transmitting a signal containing information regarding the motion disorder.

The apparatus may further include a display unit displaying the information regarding the motion disorder received from the second communication unit.

The motion characteristic value of the distal end may include velocity, acceleration, or distance moved.

### BRIEF DESCRIPTION OF THE DRAWINGS

The above and other aspects, features, and advantages of the present invention will become apparent from the detailed description of the following embodiments in conjunction with the accompanying drawings, in which:

FIG. 1 is a block diagram of an apparatus for early detection of paralysis based on motion sensing according to one embodiment of the present invention;

FIG. 2 is a view of one example of a sensor worn on a user wrist;

FIG. 3 is a view showing motion of a left wrist; and

FIG. 4 is a view showing motion of a right wrist.

It should be understood that the drawings are not necessarily to scale and that the embodiments disclosed herein are sometimes illustrated by fragmentary views. In certain instances, details which are not necessary for an understanding of the present invention or which render other details difficult to perceive may have been omitted. It should be understood that the invention is not necessarily limited to the particular embodiments illustrated herein. Like numbers utilized throughout the various figures designate like or similar parts or structure.

### DETAILED DESCRIPTION

Exemplary embodiments of the invention will now be described in detail with reference to the accompanying drawings. It should be understood that the present invention is not limited to the following embodiments and may be embodied in different ways, and that the embodiments are given to provide complete disclosure of the invention and to provide thorough understanding of the invention to those skilled in the art. The scope of the invention is limited only by the accompanying claims and equivalents thereof. Descriptions of details apparent to those skilled in the art will be omitted for clarity.

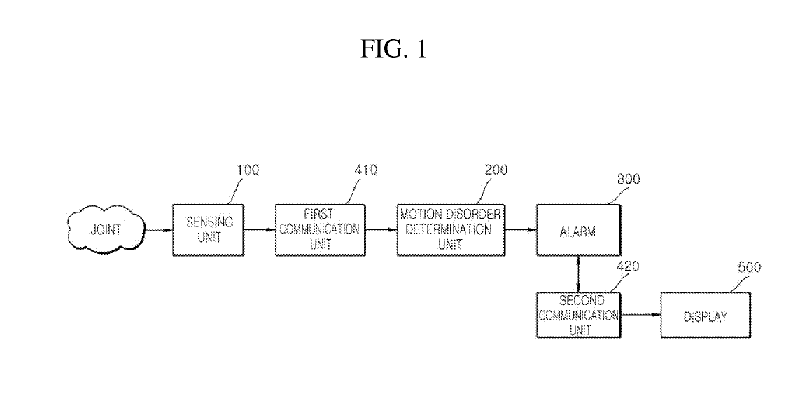

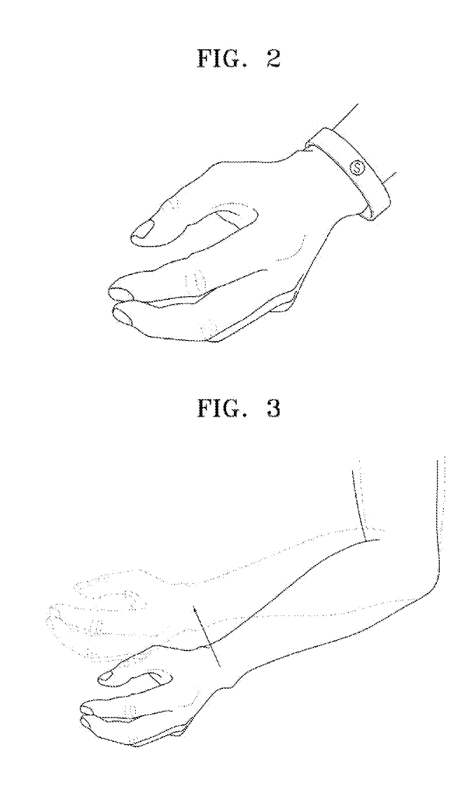

FIG. 1 is a block diagram of an apparatus for early detection of paralysis based on motion sensing according to one embodiment of the present invention.

Referring to FIG. 1, an apparatus for early detection of paralysis according to this embodiment includes: a sensing unit 100; and a motion disorder determination unit 200 which detects a motion disorder of a patient based on difference in extension and flexion of right and left distal ends.

First, the sensing unit 100 will be described.

The sensing unit 100 serves to detect a motion characteristic value of a distal end of upper or lower limbs. Here, the term “upper limbs” refer to both arms of a patient body, and the term “lower limbs” refer to both legs of the patient body. In addition, the term “distal end(s)” refers to ends of four limbs, such as wrists and ankles of a patient body, performing extension and flexion. Here, the term “motion of the distal end” refers to extension, flexion, movement, or rotation thereof.

Here, the term “extension” refers to a straightening action of the distal end, and the term “flexion” refers to a folding action of the distal end.

The sensing unit 100 includes a plurality of sensors, each of which is attached to the distal end of the upper or lower limbs. The sensors of the sensing unit 100 may measure a motion characteristic value thereof by detecting motion of the distal end.

Here, the motion characteristic value refers to a velocity, acceleration or moved distance depending on motion of the distal end. The acceleration represents a change in velocity per unit time, and the moved distance may be calculated using the velocity and time.

The sensing unit 100 detects motion of the distal end, extracts an electrical signal from the motion characteristic value thereof, and transmits the signal to the motion disorder determination unit 200 through a first communication unit 410 described below.

In one embodiment, an accelerometer is used as the sensor attached to the distal end. Here, the accelerometer is a generic term for sensors which measure motion of the distal end based on motion of a patient body. Such an accelerometer is attached to each of distal ends of four limbs. These sensors may be worn on distal portions of a patient body, such as the wrists or the ankles, in the form of watches or anklets.

The accelerometer (S) receives analog information about a degree of motion of the wrist rotating about the elbow as a rotating center. The received analog information about the degree of motion of the wrist is transmitted to an analog-to-digital (A/D) converter, which in turn converts the analog information into a digital signal.

The motion disorder determination unit 200 described below may determine the degree of folding or straightening of a patient's elbow based on sensing results of the accelerometer (S) transmitted after conversion into a digital signal.

Here, a tilt sensor may be placed to measure motion of the wrist in 2-axis or 3-axis directions. Although the accelerometer has been illustrated as the sensor of the sensing unit 100 in the above description, the present invention is not limited thereto, and various other types of sensors, such as tilt sensors, gyroscopes, and the like, may be used.

Next, three exemplary embodiments of the motion disorder determination unit 200 will be described.

### First Embodiment

The motion disorder determination unit 200 receives a motion characteristic value of each distal end of a patient body extracted by the sensing unit 100, and determines whether a difference between the motion characteristic values of the right and left distal ends exceeds a reference value.

That is, the motion disorder determination unit 200 determines whether a patient wearing the sensing unit 100 begins to show a hemi-paralysis symptom, which is an early-stage symptom of a stroke, based on a difference in motion between left limbs and right limbs of a patient body. For example, the motion disorder determination unit 200 determines whether a difference in angular velocity between a left wrist and a right wrist of a patient falls within a normal range depending on the degree of extension and flexion thereof.

Here, the term “reference value” refers to a limit value of difference in motion which can occur without paralysis. That is, a difference in motion below the reference value means normal motion, and a difference in motion exceeding the reference value means that a patient has suffered a stroke.

That is, a difference in motion between the distal ends less than the reference value means normal motion without hemi-paralysis, and a difference in motion between the distal ends exceeding the reference value means that a stroke occurs in a patient.

The reference value may be set depending on motion characteristics of a patient body, such as walking, running, sitting, and the like.

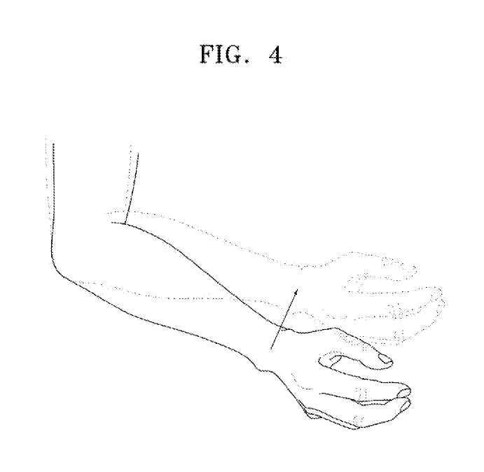

Referring to FIGS. 3 and 4, determination on a motion disorder a patient will now be described in more detail.

FIG. 3 is a view showing motion of a left wrist, and FIG. 4 is a drawing showing motion of a right wrist.

In FIGS. 3 and 4, as one example of the distal end, the left wrist and the right wrist are shown. It can be understood that a motion disorder of a patient may be determined by detecting motion of other distal ends instead of the wrist.

When flexion of a left elbow occurs in response to a certain motion of a patient body, velocity and acceleration, which can be represented by an arrow in FIG. 3, are generated. By measuring the velocity and the acceleration represented by the arrow in FIG. 3 at regular intervals for a predetermined period of time, the sum of motion of the left wrist corresponding to the certain motion of the patient body may be measured.

As shown in FIG. 4, when flexion of a right elbow occurs in response to a certain motion of the patient body, a velocity and acceleration, which can be represented by an arrow in FIG. 4, are generated.

By measuring the velocity and the acceleration represented by the arrow in FIG. 4 at regular intervals for a predetermined period of time, the sum of motion of the right wrist corresponding to the certain motion of the patient body may be measured.

In response to a certain motion characteristic of the patient body, when a difference between the sum of motion of the left wrist and the sum of motion of the right wrist for a predetermined time exceeds the reference value, the motion disorder determination unit 200 determines a motion disorder and transmits information regarding the motion disorder to an alarm unit 300.

Here, since the patient encountering a stroke becomes paralyzed at one side of the upper and lower limbs, the fact that motion of the distal ends at one side of the patient is significantly slowed down is used for detection of a stroke at an early stage.

### Second Embodiment

The motion disorder determination unit 200 receives a motion characteristic value of each of the distal ends extracted by the sensing unit 100, and determines whether a difference between the sum of motion characteristic values of each of the distal ends for a unit time and the sum of motion characteristic values of each of the distal ends for a subsequent unit time exceeds a reference value.

Now, this operation will be described in more detail.

A patient completely recovered from initial mild paralysis shows normal motion of four limbs. However, since there is a strong possibility that such a patient will suffer a second stroke, intensive monitoring of the patient is needed.

Therefore, reference data of individual patients in a normal state on a motion characteristic value per unit time is collected, and data measured over time is compared with the reference data, thereby enabling early and sensitive detection of paralysis due to the occurrence of a stroke.

Since the characteristics of extension and flexion change every moment, the motion disorder determination unit 200 sensitively determines the occurrence of paralysis by comparing the sum of the characteristic values of extension and flexion for a unit time (for example, 30 minutes) to the sum of the characteristic values of extension and flexion for a previous unit time.

Here, the reference value refers to a limit value of difference in motion per unit time, which can be made without paralysis. That is, a difference in each motion less than the reference value means normal motion, and a difference in each motion exceeding the reference value means that a patient has suffered a stroke.

The reference value may be set depending on motion characteristics of a patient body, such as walking, running, sitting, and the like.

### Third Embodiment

The motion disorder determination unit 200 receives a motion characteristic value of each of the distal ends extracted by the sensing unit 100, and determines whether the sum of motion characteristic values of each of the distal ends detected by the sensing unit for a unit time is less than a reference value.

That is, the motion disorder determination unit 200 may detect the occurrence of paralysis by detecting that the motion characteristic value of a patient for a certain period of time does not reach the reference value. Since a normal person feels uncomfortable when the joint is not moved for a certain period of time, the person without paralysis unconsciously moves the body slightly.

If there is no movement in four limbs of a patient for a certain period of time, for example 30 minutes, 1 hour, or the like, it can be determined that the patient suffers paralysis.

Here, the reference value refers to a minimum value among sums of the motion characteristic values of each of the distal ends, which naturally occur for a unit time based on normal biomechanical motion.

That is, the sum of the motion characteristic values for a unit time exceeding the reference value means that the patient shows normal motion without paralysis, and the sum of the motion characteristic values for a unit time less than the reference value means that the patient suffers paralysis.

Next, the alarm unit 300, the second communication unit 420 and the display unit will be described.

The alarm unit 300 serves to warn a doctor or nurse of a motion disorder of a patient when the motion disorder is determined by the motion disorder determination unit 200. The alarm unit 300 may include a data processing module in order to transmit data containing information regarding the motion disorder to a doctor or nurse.

The second communication unit 420 includes a wired or wireless transceiver module and transmits information regarding the motion disorder of a patient to a doctor or nurse at a remote place. The display unit 500 displays the information regarding the motion disorder of the patient received from the second communication unit 420 on a screen so that a doctor or nurse can visually check motion conditions of the patient. In addition to the alarm unit 300, the second communication unit 420 and the display unit 500, the apparatus may further include components performing various additional functions.

As such, according to the present invention, the apparatus includes a motion disorder determination unit which determines whether patient motion is abnormal based on a difference in motion characteristics of distal ends, such as left and right wrists and ankles, and the like, for a certain period of time, thereby enabling early detection on the occurrence of paralysis due to stroke.

In addition, the apparatus includes an alarm unit warning of a patient motion disorder, a second communication unit transmitting real-time information regarding the motion disorder to a doctor, and a display unit displaying the information, thereby providing an advantageous effect that the patient can be rapidly treated by the doctor.

One embodiment of the present invention allows to detect paralysis suffered by a patient during his or her sleeping.

Although some embodiments have been described herein, it should be understood by those skilled in the art that these embodiments are given by way of illustration only, and that various modifications, variations, and alterations can be made without departing from the spirit and scope of the invention. Therefore, the scope of the invention should be limited only by the accompanying claims and equivalents thereof.

Thus, there has been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. Many changes, modifications, variations and other uses and applications of the present constructions will, however, become apparent to those skilled in the art after considering the specification and the accompanying drawings. All such changes, modifications, variations and other uses and applications which do not depart from the spirit and scope of the invention are deemed to be covered by the invention and the scope of the present disclosure is not intended to be limited solely to the embodiments shown herein. All structural and functional equivalents to the elements of the various embodiments described throughout this disclosure that are known or later come to be known to those of ordinary skill in the art are expressly incorporated herein by reference and are intended to be encompassed by this disclosure.

What is claimed is:

1. An apparatus for early detection of paralysis based on motion sensing, comprising:
a sensing part configured to detect motion of at least one left distal end portion and at least one right distal end portion of upper or lower limbs of a patient; and
a motion disorder determination unit configured to determine whether a difference between a sum of motion characteristic values of the at least one left distal end portion and a sum of motion characteristic values of the at least one right distal end portion exceeds a predetermined reference value wherein both the motion characteristic values of the at least one left distal end portion and the motion characteristic values of the at least one right distal end portion are determined based on motion detected by the sensing part for a certain period of time.

2. The apparatus according to claim 1, wherein the sensing part comprises a first communication unit for transmitting information of detected motion of the at least one left distal end and the at least one right distal end.

3. The apparatus according to claim 1, further comprising: an alarm unit for warning of a motion disorder when the motion disorder determination unit determines that the difference between the sum of the motion characteristic values of the at least one left distal end and that of the at least one right distal end exceeds the predetermined reference value.

4. The apparatus according to claim 3, wherein the alarm unit comprises a second communication unit for transmitting a signal containing information regarding the motion disorder.

5. The apparatus according to claim 4, further comprising: a display unit configured to display the information regarding the motion disorder received from the second communication unit.

6. The apparatus according to claim 1, wherein the motion characteristic value of the distal ends comprises any one of velocity, acceleration, and distance moved.

7. The apparatus according to claim 1, wherein the at least one distal end corresponds to the at least one right distal end.

8. The apparatus according to claim 1, wherein the sensing part includes at least one gyroscope.

9. An apparatus for early detection of paralysis based on motion sensing, comprising:
a sensing part configured to detect motion of at least one distal end of upper or lower limbs; and
a motion disorder determination unit configured to determine whether a difference between a sum of motion characteristic values of the at least one distal end detected by the sensing part for a first predetermined time period and a sum of motion characteristic values of the at least one distal end detected by the sensing part for a subsequent second predetermined time period exceeds a predetermined reference value.

10. The apparatus according to claim 9, wherein the sensing part comprises a first communication unit for transmitting information of motion of the at least one distal end.

11. The apparatus according to claim 9, further comprising: an alarm unit for warning of a motion disorder when the motion disorder determination unit determines that the difference between the sum of the motion characteristic values of the at least one distal end detected by the sensing part for a third predetermined time period and the sum of motion characteristic values of the at least one distal end detected by the sensing part for a subsequent fourth predetermined time period exceeds the predetermined reference value.

12. The apparatus according to claim 11, wherein the alarm unit comprises a second communication unit for transmitting a signal containing information regarding the motion disorder.

13. The apparatus according to claim 12, further comprising: a display unit configured to display the information regarding the motion disorder received from the second communication unit.

14. The apparatus according to claim 9, wherein the motion characteristic value of the distal end comprises any one of velocity, acceleration, and distance moved.

15. An apparatus for early detection of paralysis based on motion sensing, comprising:
a sensing part configured to detect motion characteristic values of at least one distal end of upper or lower limbs; and
a motion disorder determination unit configured to determine whether a difference between a sum of motion characteristic values of the at least one distal end detected by the sensing part for a unit time and a sum of motion characteristic values of the at least one distal end detected by the sensing part for a subsequent unit time exceeds a predetermined reference value.

16. The apparatus according to claim 15, further comprising: an alarm unit for warning of a motion disorder when the motion disorder determination unit determines that the sum of the motion characteristic values of the at least one distal end detected by the sensing part for a unit time is less than the predetermined reference value.

17. The apparatus according to claim 15, wherein the alarm unit comprises a second communication unit for transmitting a signal containing information regarding the motion disorder.

18. The apparatus according to claim 15, further comprising: a display unit configured to display the information regarding the motion disorder received from the second communication unit.

19. The apparatus according to claim 15, wherein the motion characteristic value of the distal end comprises any one of velocity, acceleration, and distance moved.

20. The apparatus according to claim 15, wherein the sensing part includes at least one gyroscope.
